(19) United States
(12) Patent Application Publication
(10) Pub. No.: US 2018/0318455 A1
(43) Pub. Date: Nov. 8, 2018

(54) COMPLEXES BETWEEN BLOCK POLYMERS AND IONS AS CONTRAST AGENTS FOR MEDICAL IMAGING

(71) Applicants: CENTRE NATIONAL DE LA RECHERCHE SCIENTIFIQUE (CNRS), Paris (FR); UNIVERSITE PAUL SABATIER TOULOUSE III, Toulouse (FR)

(72) Inventors: Christophe MINGOTAUD, Toulouse (FR); Jean-Daniel MARTY, Lacroix Falgarde (FR); Camille FRANGVILLE, Guyancourt (FR); Daniel THALHAM, Gainesville, FL (US)

(73) Assignees: CENTRE NATIONAL DE LA RECHERCHE SCIENTIFIQUE (CNRS ), Paris (FR); UNIVERSITE PAUL SABATIER TOULOUSE III, Toulouse (FR)

(21) Appl. No.: 15/773,471

(22) PCT Filed: Nov. 4, 2016
(86) PCT No.: PCT/EP2016/076721

§ 371 Date: May 3, 2018

(30) Foreign Application Priority Data
Nov. 5, 2015 (FR) 1560635

(51) Int. Cl. A61K 49/12; A61K 51/06; A61K 9/00; A61K 49/00

(52) U.S. Cl. CPC A61K 49/126; A61K 51/065; A61K 49/0002; A61K 9/0053; A61K 9/0019

(57) ABSTRACT

The invention relates to a complex between block polymers comprising a first hydrophilic block and a second block comprising at least one ionised function, and optionally carrying at least one additional chemical or biochemical group or comprising a third polymer block, and mixtures of said polymers, and ions selected from the elements Cu, Ga, Sr, Bi, Se, Y, lanthanide, Pb, Te, Zn, Zr at different degrees of ionisation and the mixtures thereof, more particularly for the use thereof in medical imaging, and to a production method and a physiologically acceptable composition containing said complex.

The present invention relates to complexes between (1) block polymers comprising a hydrophilic part and an ionized part with (2) ions useful in medical imaging, these complexes being usable as contrast agents.

Magnetic resonance imaging (MRI) is an effective medical diagnostic tool whose efficient implementation requires the use of contrast agents These contrast agents are broken down into two families: positive contrast agents that reduce the longitudinal relaxation times of proton spins and negative contrast agents that affect their transverse relaxation time. The first improve the contrast of areas rich in contrast agents and have proven effective in biological applications (Bottomley et al. (1984); Oostendorp et al. (2010); Tseng et al. (2010)).

Gadolinium molecular complexes are particularly interesting candidates to obtain such positive contrast agents. However, due to the many interferences with the biological environment, free Gd³⁺ ions have significant toxicity. In order to reduce this toxicity, gadolinium chelators have been developed to limit the free circulation of free Gd3³⁺ in the body, which has led to the marketing of Dotarem® (DOTA) and Magnevist® (DTPA). However, these commercial contrast agents have certain disadvantages (Abraham et al. (2008)) since:

i) they do not completely eliminate ion exchanges with the biological environment, which can lead to long-term toxicity of these components, and

ii) they are rapidly eliminated from the bloodstream which, for optimal use as a contrast agent, requires them to be injected in a relatively large quantity which, in turn, increases their toxicity.

Thus, the discovery of highly-effective contrast agents, i.e., having significant relaxivity values allowing high contrasts to be obtained for low concentrations, remains a challenge that is the subject of intense research efforts. For this purpose, a strategy that is being particularly considered consists of grafting gadolinium chelates onto macromolecular systems (US 2007/0154398), which improves the relaxation properties and slows the rotational movements of these contrast agents. However, obtaining such systems requires many synthesis steps, which is often incompatible with industrial production.

There is therefore a real need for new contrast agents, in particular gadolinium based, overcoming the disadvantages of the contrast agents known in the art encountered in MRI.

It is likewise in other types of imaging relying on the use of radioactive isotopes (α, β or γ emitters). This type of contrast agent should ensure an excellent biocompatibility as well as easy production of these agents (minimal synthesis or purification steps).

Moreover, there is a great need concerning the development of new contrast agents for multimodal imaging contrast, a same compound strengthening the signal in several imaging techniques.

The inventors of the present invention thus discovered that it was possible to prepare such contrast agents by complexation of ions useful in medical imaging, and in particular gadolinium ions, with block polymers comprising a first hydrophilic part and a second ionized part (or ionizable part that will be ionized during the preparation process). The first part of the polymer allows stabilizing the complex in solution in order to obtain stable colloidal solutions of these complexes while improving the stealth of the system. It also allows considerably reducing the toxicity of the systems. The second part of the polymer generates non-specific interactions with the ions, which play the role of structural bridges between copolymers. Such complexes have the additional advantage of being able to be prepared by a very simple process involving the simple mixing of block polymers with the ions; the complexes form spontaneously. Thus, ions, in addition to their role of contrast agent, are also active in their own formulation by complexation with block polymers. Moreover, the polymers used can be commercially-available polymers, thus avoiding the development of specific ligands for the ions in question, as in the past.

The present invention therefore relates to a complex between:
block polymers comprising a first hydrophilic block and a second block comprising at least one ionized function, and optionally carrying one or more additional chemical or biochemical groups (fluorescent groups, specific biochemical ligands, etc.) or including a third block polymer, or mixtures of these polymers, and
ions chosen from among the elements Cu, Ga, Sr, Bi, Sc, Y, lanthanide (e.g., Eu or Gd), Pb, Tc, Zn, Zr at different degrees of ionization and mixtures thereof, for use in medical imaging, particularly to visualize a pathologic structure, such as a tumor.

These complexes are particularly useful as contrast agents.

In the sense of the present invention, “medical imaging” means a method for acquisition and restitution of images of the animal body, and more particularly the human body. Such a method can be based on different physical phenomena such as x-ray absorption, nuclear magnetic resonance, fluorescence or radioactivity (α, β or γ). It particularly permits indirectly visualizing the anatomy, physiology or metabolism of the animal, in particular human, body and can therefore be useful as a diagnostic tool.

In the sense of the present invention, “contrast agent” means a substance that artificially increases contrast, allowing visualization of an anatomical structure (for example, an organ) or pathological structure (for example, a tumor) that naturally has little or no contrast, and which would otherwise be difficult to distinguish, especially relative to adjacent tissues.

Block Polymers

Block polymers present in complexes according to the present invention comprise:
a first hydrophilic block, and
a second block comprising at least one ionized function.

It may also:
comprise a third block polymer and/or
carry one or more additional chemical or biochemical groups (fluorescent groups, specific biochemical ligands, etc.).

In the sense of the present invention, “block polymer” (also called “block copolymer”), means a polymer (and more particularly a copolymer) made up of at least two blocks bound covalently, each block resulting from the polymerization of a monomer or mix of monomers, the monomer or mix of monomers being different from one block to another. Thus, the block polymer PEO-PAA comprises a first block, PEO (poly(ethylene oxide)), resulting from the polymerization of ethylene oxide covalently bound to a second block, PAA (poly(acrylic acid)), resulting from the polymerization of acrylic acid.

In the context of the present invention, this will more particularly be a diblock polymer (comprising two different blocks), triblock polymer (comprising three different blocks) or a mixture thereof, and advantageously a diblock polymer or possibly a mixture of diblock polymers.

In the sense of the present invention, “hydrophilic block” means a block comprising hydrophilic motifs. Such a block will therefore be obtained by polymerization of a monomer or mixture of monomers comprising hydrophilic motifs that remain after polymerization, such as oxygen or nitrogen-containing motifs, and particularly ethers or amides. Advantageously, this block will not comprise an ionizable function.

In the sense of the present invention, “ionizable function” means a function that can be ionized to give a positively or negatively charged species. A function can be ionized more particularly by action of an acid or base, particularly by loss or gain of one or more H⁺ protons. Ionizable functions are, for example, carboxylic acid (—CO₂H), hydroxyl (—OH), phosphonic acid (—P(O)(OH)₂), phosphate (—OP(O)(OH)₂), sulfonic acid (—S(O)₂OH) or sulfate (—OS(O)₂OH) functions.

The hydrophilic block can more particularly be:
a PEO (poly(ethylene oxide)) block, also called PEG (poly(ethylene glycol)), such a block comprising ether functions as hydrophilic motifs in the polymer chain itself,
a PNIPAM (poly(N-isopropylacrylamide)) block, such a block comprising amide (CONH) functions as hydrophilic motifs in the polymer chain,
a polyvinylpyrrolidone (PVP) block, such a block comprising amide (CONH) functions as hydrophilic motifs in the polymer chain,
a polyvinylcaprolactam (PVCL) block, such a block comprising amide (NCO) functions as hydrophilic motifs in the polymer chain,
a polydimethylacrylamide (PDMA) block, such a block comprising amide (NCO) functions as hydrophilic motifs in the polymer chain, or
   a mixture thereof.

The hydrophilic block can more particularly be:
a PEO (poly(ethylene oxide)) block, also called PEG (poly(ethylene glycol)), such a block comprising ether functions as hydrophilic motifs in the polymer chain itself,
a PNIPAM (poly(N-isopropylacrylamide)) block, such a block comprising amide (CONH) functions as hydrophilic motifs in the polymer chain,
a polyvinylpyrrolidone (PVP) block, such a block comprising amide (CONH) functions as hydrophilic motifs in the polymer chain,
a polyvinylcaprolactam (PVCL) block, such a block comprising amide (NCO) functions as hydrophilic motifs in the polymer chain, or
   a mixture thereof.

The number average molar mass of the hydrophilic block is advantageously comprised between 3,000 g·mol⁻¹ and 50,000 g·mol⁻¹, particularly between 5,000 g·mol⁻¹ and 20,000 g·mol⁻¹.

The hydrophilic block of the block polymer according to the present invention, which will particularly be found on the external part of the complex, allows stabilizing the complex in aqueous solution in order to obtain a stable colloidal solution, as well as to ensure its biocompatibility and optimize its maintenance in biological fluids.

This hydrophilic block may optionally carry one or more additional chemical groups such as a fluorophore (coumarin, etc.) or one or more biochemical groups of interest (peptide, protein, sugar, etc.).

In the sense of the present invention, “ionized function/form” means a charged function/form, and more particularly negatively charged.

In the sense of the present invention, “block comprising at least one ionized function” means a block comprising at least one negatively-charged function (functional group), insofar as the ions used for complexation are cations. Such a block will therefore be obtained by polymerization of a monomer or mix of monomers comprising ionizable or already-ionized functions, particularly in the form of a salt, and which remain after polymerization.

The ionized functions that can be present on this block are particularly chosen from among —CO₂⁻ (carboxylate), —O⁻ (alcoholate), —PO₃²⁻ (phosphonate), —OPO₃²⁻ (phosphate), —SO₃⁻ (sulfonate) and —OSO₃⁻ (sulfate) groups and combinations thereof. It can particularly be —CO₂⁻ or —PO₃²⁻ functions or a combination thereof.

The block comprising at least one ionized function can be, in particular, a PAA (poly(acrylic acid)) block, a PVPA (poly(vinylphosphonic acid)) block, or a mixture thereof, in the ionized form, advantageously a PAA block in an ionized form.

The number average molar mass of the block comprising at least one ionized function is advantageously comprised between 1,000 g·mol⁻¹ and 20,000 g·mol⁻¹, particularly between 3,000 g·mol⁻¹ and 6,000 g·mol⁻¹.

This block comprising negative charges will allow the formation of a complex by electrostatic interaction of several block polymer chains and several ions bearing positive charges.

This block comprising at least one ionized function can also potentially comprise additional functions that can also interact non-electrostatically (for example, dative bonds) with ions carrying positive charges.

This block comprising at least one ionizing function can also optionally carry additional functions such as a fluorophore (coumarin, etc.).

The ratio between the mass of the hydrophilic blocks and comprising at least one ionized function must advantageously be greater than around 0.5 to ensure the formation of colloids of well-defined sizes.

The block polymer can particularly be a polymer in the ionized form of PEO-PAA, PVCL-PAA, PNIPAM-PAA, PDMA-PAA or a mixture thereof.

The block polymer can particularly be a polymer in the ionized form of PEO-PAA, PVCL-PAA, PNIPAM-PAA, or a mixture thereof.

The use of a single type of block polymer, or, on the contrary, a mixture of block polymers, in the complex according to the invention can also be envisaged. These polymers can also be mixed with a small quantity of functional homopolymers that can interact with the ions.

A polymer (e.g., PAA-PDMA) carrying a fluorescent group (e.g., a coumarin), such as PAA-PDMA-coumarin, may be present, particularly in a proportion of 1 to 10% by weight relative to the total polymer weight.

The number average molar mass of the block polymer is advantageously comprised between 2,000 g·mol⁻¹ and 70,000 g·mol⁻¹, particularly between 8,000 g·mol⁻¹ and 26,000 g·mol⁻¹.

The mass ratio between the hydrophilic block and the block comprising at least one ionized function is advantageously comprised between 0.5 and 100, particularly between 1 and 3.

Ions

The ions used in the complexes according to the present invention are ions useful in medical imaging. They therefore have a dual role since they act both as a contrast agent and as an actor in their own formulation by creating interactions with the ionized part of the block polymers.

The ions used in the context of the present invention are cations and more particularly have a charge greater than or equal to 2+. Indeed, having ions with at least two charges allows creating at least two interactions with the block polymers according to the invention so as to create a colloidal assembly.

Ions are chosen from among the elements Cu, Ga, Sr, Bi, Sc, Y, lanthanide (e.g., Eu or Gd), Pb, Tc, Zn, Zr with different degrees of ionization and mixtures thereof, and can more particularly be chosen from among the elements Cu, Eu, Gd, Tc, Zr at different degrees of ionization and mixtures thereof.

The ions can then more particularly be chosen from among Cu²⁺, Ga³⁺, Sr²⁺, Bi³⁺, Sc³⁺, Y³⁺, Eu³⁺, Gd³⁺, Pb²⁺, Tc⁵⁺, Zn²⁺, Zr⁴⁺ and mixtures thereof, particularly chosen from among Cu²⁺, Eu³⁺, Gd³⁺, Tc⁵⁺, Zr⁴⁺ and mixtures thereof, in particular Cu²⁺, Eu³⁺, Gd3+ and mixtures thereof, and advantageously chosen from among Gd³⁺, Cu²⁺ and mixtures thereof. It can particularly be Gd³⁺.

As for block polymers, the use of a single type of ion in the complex according to the invention, or on the contrary, a mixture of ions, can also be envisaged.

Complex

The complex according to the invention results from the complexation of several block polymer chains and several ions. The fact that the ions have at least two positive charges allows a three-dimensional structure to be created, comprising the blocks comprising at least one ionized function in interaction with the ions in the center and the hydrophilic blocks at the periphery. Such a complex is obtainable by the preparation process described below in the description.

Complexes according to the present invention are thus found in the form of particles that can have a size comprised between 5 and 100 nm, in particular between 10 and 50 nm. This size can be measured by dynamic light scattering.

These complexes can be in the form of a colloidal aqueous solution.

Medical Imaging

Complexes according to the present invention can be used in different types of medical imaging according to the type of ions present, such as:
magnetic resonance imaging (MRI) for Gd³⁺ ions,
fluorescence imaging for lanthanide ions, for example based on europium, and
scintigraphic imaging (including positron emission tomography (PET) and alpha emission imaging) for Cu, Pb, Ga, Bi, Tc and Zr-based ions, involving the presence of a radioisotope of the ions used (e.g., for technetium, presence of the ^{99m}Tc radioisotope).

The choice of ions and polymers allows creating complexes that can be used simultaneously for several types of imaging.

The present invention also concerns the use of a complex according to the present invention defined above as a contrast agent for medical imaging, particularly as defined above.

The present invention also concerns the use of a complex according to the present invention defined above for the preparation of a physiologically-acceptable composition, particularly a diagnostic composition, useful, in particular, for medical imaging, particularly as defined above.

The present invention also concerns a medical imaging method, particularly as defined above, comprising the administration to a subject in need of an effective dose of a complex according to the present invention defined above.

The present invention also relates to a physiologically-acceptable composition, more particularly a diagnostic composition, comprising a complex according to the present invention in a physiologically-acceptable medium.

According to one particular embodiment, the complex of the physiologically-acceptable composition is not:
complexes between Zn²⁺ ions and poly(acrylic acid)-polyacrylamide (PAA-PAM) diblock polymers in the ionized form for which the poly(acrylic acid) block has a number average molecular weight of 1,000, 3,000, or 5,000 g·mol⁻¹ and the polyacrylamide block as a number average molecular weight of 10,000, 15,000, or 30,000 g·mol⁻¹ respectively,
complexes between La³⁺ ions and poly(acrylic acid)-polyacrylamide (PAA-PAM) diblock polymers in an ionized form for which the polyacrylamide block has a number average molecular weight of 10,000 g·mol⁻¹ and the poly(acrylic acid) block has a number average molecular weight of 1,000, 3,000 or 6,000 g·mol⁻¹; or the polyacrylamide block has a number average molecular weight of 11,100 g·mol⁻¹ and the poly(acrylic acid) block has a number average molecular weight of 2,800 g·mol⁻¹; or the polyacrylamide block has a number average molecular weight of 30,000 g·mol⁻¹ and the poly(acrylic acid) block has a number average molecular weight of 3,000 or 5,000 g·mol⁻¹; or the polyacrylamide block has a number average molecular weight of 60,000 g·mol⁻¹ and the poly(acrylic acid) block has a number average molecular weight of 5,000 g·mol⁻¹, or
complexes between La³⁺ ions and poly(acrylic acid)-poly(2-hydroxyethyl acrylate) (PAA-PHEA) diblock polymers in the ionized form for which the poly(acrylic acid) block has a number average molecular weight of 1,900, or 2,800 g·mol⁻¹ and the poly(2-hydroxyethyl acrylate) block has a number average molecular weight of 8,200, or 11,100 g mol⁻¹, respectively,

In the present invention, “physiologically acceptable” means what is useful in the preparation of a composition intended to be administered to an animal, including human, which is generally safe, non-toxic and neither biologically nor otherwise undesirable.

The compositions according to the present invention are intended more particularly to be administered parenterally, particularly intravenously or orally.

The compositions according to the invention for parenteral administration will therefore advantageously be in a liquid form, and more particularly in the form of aqueous solutions, suspensions or emulsions. These compositions can also contain sodium chloride, a physiologically-acceptable acid or base (to adjust the pH), a preservative, a dispersion agent, a wetting agent or a combination thereof.

Moreover, these compositions will advantageously be in a sterile form and isotonic to the blood plasma (i.e., with an osmotic pressure close to that of blood) in the case of intravenous administration.

The compositions according to the invention for oral administration can be in the form of tablets, capsules, granules, powders, solutions, suspensions or emulsions. These compositions can also contain pharmaceutically-acceptable excipients conventionally used for this type of composition and well known to those skilled in the art.

The present invention also relates to a complex between:
block polymers comprising a first hydrophilic block and a second block comprising at least one ionized function, and optionally carrying one or more additional chemical or biochemical groups (fluorescent groups, specific biochemical ligands, etc.) or including a third block polymer, or mixtures of these polymers, particularly such as defined previously, and
ions chosen from among the elements Cu, Ga, Sr, Bi, Sc, Y, lanthanide (e.g., Eu or Gd), Pb, Tc, Zn, Zr at different degrees of ionization and mixtures thereof, particularly as defined previously,
   with the exception of:
complexes between Zn²⁺ ions and poly(acrylic acid)-polyacrylamide (PAA-PAM) diblock polymers in the ionized form for which the poly(acrylic acid) block has a number average molecular weight of 1,000, 3,000, or 5,000 g·mol⁻¹ and the polyacrylamide block has a number average molecular weight of 10,000, 15,000, or 30,000 g·mol⁻¹ respectively,
complexes between La³⁺ ions and poly(acrylic acid)-polyacrylamide (PAA-PAM) diblock polymers in an ionized form for which the polyacrylamide block has a number average molecular weight of 10,000 g·mol⁻¹ and the poly(acrylic acid) block has a number average molecular weight of a 1,000, 3,000 or 6,000 g·mol⁻¹; or the polyacrylamide block has a number average molecular weight of 30,000 g·mol⁻¹ and the poly(acrylic acid) block has a number average molecular weight of 3,000 or 5,000 g·mol⁻¹; or the polyacrylamide block has a number average molecular weight of 60,000 g·mol⁻¹ and the poly(acrylic acid) block has a number average molecular weight of 5,000 g·mol⁻¹, and
complexes between La³⁺ ions and poly(acrylic acid)-poly(2-hydroxyethyl acrylate) (PAA-PHEA) diblock polymers in the ionized form for which the poly(acrylic acid) block has a number average molecular weight of 1,900, or 2,800 g·mol⁻¹ and the poly(2-hydroxyethyl acrylate) block has a number average molecular weight of 8,200, or 11,100 g·mol⁻¹, respectively.

Advantageously, the complex according to the invention is not a complex between La³⁺ ions and poly(acrylic acid)-polyacrylamide (PAA-PAM) diblock polymers in the ionized form for which the polyacrylamide block has a number average molecular weight of 11,100 g·mol⁻¹ and the poly(acrylic acid) block has a number average molecular weight of 2,800 g·mol⁻¹.

The polymers thus excluded are already described in the art, no use as contrast agent in medical imaging being reported (Bouyer et al. (2003); Sanson et al. (2012); Tarasov et al. (2013)).

According to one particular embodiment of the invention, the complex according to the invention will not be:
a complex between La³⁺ ions and poly(acrylic acid)-polyacrylamide (PAA-PAM) diblock polymers in the ionized form for which the poly(acrylic acid) block has a number average molecular weight of 3,000 g·mol⁻¹ and the polyacrylamide block has a number average molecular weight of 15,000 g·mol⁻¹,
a complex between La³⁺ ions and poly(acrylic acid)-poly(2-hydroxyethyl acrylate) (PAA-PHEA) diblock polymers in the ionized form for which the poly(acrylic acid) block has a number average molecular weight of 900 g·mol⁻¹ and the poly(2-hydroxyethyl acrylate) block has a number average molecular weight of 9,200 g·mol⁻¹.

According to another particular embodiment of the invention, the complex according to the invention will not be:
a complex between Zn²⁺ ions and poly(acrylic acid)-polyacrylamide (PAA-PAM) diblock polymers in an ionized form,
a complex between La³⁺ ions and poly(acrylic acid)-polyacrylamide (PAA-PAM) diblock polymers in an ionized form,
a complex between La³⁺ ions and poly(acrylic acid)-poly(2-hydroxyethyl acrylate) (PAA-PHEA) diblock polymers in an ionized form.

The preferred block polymers and ions will more particularly be such as indicated previously. According to one particular embodiment, the ions will not be Zn²⁺ or La³⁺ ions.

The present invention also relates to a process for the preparation of complexes according to the present invention, such as defined previously, comprising the complexation reaction between:
at least one block polymer comprising a first hydrophilic block and a second block comprising at least one ionized (e.g., in the form of a salt) or ionizable function, and optionally carrying one or more additional chemical or biochemical groups (fluorescent groups, specific biochemical ligands, etc.) or including a third block polymer, and
at least one salt of an ion chosen from among the elements Cu, Ga, Sr, Bi, Sc, Y, lanthanide (e.g., Eu or Gd), Pb, Tc, Zn, and Zr at different degrees of ionization.

The block polymer could more particularly be in the form of a salt, particularly sodium or potassium, or in a nonionized form (which could be ionized in solution, particularly under appropriate pH conditions).

The salt of an ion chosen from among the elements Cu, Ga, Sr, Bi, Sc, Y, lanthanide (e.g., Eu or Gd), Pb, Tc, Zn, and Zr at different degrees of ionization could more particularly be a nitrate, chloride or sulfate. Particularly, the Cu²⁺ salt could be Cu(NO₃)₂; the Eu³⁺ salt could be Eu(NO₃)₂; the Gd³⁺ salt could be Gd(NO₃)₂. The complexation reaction will advantageously be done in water as the solvent, advantageously at pH near physiological conditions.

The complex thus formed could be separated from the reaction medium, for example by lyophilization or used as such, in the form of a colloidal aqueous solution. However, the salt formed, for example, between the original counterion of the block polymer and the original counterion of the ions chosen from the elements Cu, Ga, Sr, Bi, Sc, Y, lanthanide (for example, Eu or Gd), Pb, Tc, Zn and Zr at different degrees of ionization also present in the colloidal aqueous solution, could be eliminated beforehand, particularly by dialysis.

The present invention is illustrated by the figures and non-limiting examples described in detail below.

### FIGURES

FIG. 1 is an illustrative diagram of the complexation reaction between the ions (triangles) and block polymers (free chains with the dark part being the hydrophilic block and the light part being the block comprising at least one ionized or ionizable function).

FIG. 2 shows the scattered intensity variation (black squares) or the average size in Z (gray squares), measured by dynamic light scattering, during preparation of complexes by reaction in water between the PEO_{6K}-b-PAA_{3K} polymer (0.1% by mass) and Gd(NO₃)₃ (increasing concentrations), depending on the ratio R between the gadolinium [Gd] concentration and the concentration of the 3 acetic groups [3.AA](present in the polymer).

FIG. 3 illustrates a colloidal solution of PEO_{6K}-b-PAA_{3K}/Gd³⁺ complexes with R=[Gd]/[3.AA]=1 observed by electron microscopy.

FIG. 4 illustrates the particle size distribution of a colloidal solution of PEO_{6K}-b-PAA_{3K}/Gd³⁺ complexes with R=[Gd]/[3.AA]=1.

FIG. 5 shows the relaxivity rates (1/T₁ in gray and 1/T₂ in black) for a colloidal solution of PEO_{6K}-b-PAA_{3K}/Gd³⁺ complexes with R=[Gd]/[3.AA]=1 which has been dialyzed.

FIG. 6 represents an image of the rat renal region before (0 min), during (during IV) and after (1 min 40 and 55 min) intravenous administration of a colloidal solution of PEO_{6K}-b-PAA_{3K}/Gd³⁺ complexes with R=[Gd]/[3.AA]=1.

FIG. 7 shows the increase in contrast ratio depending on the time after intravenous administration of a colloidal solution of PEO_{6K}-b-PAA_{3K}/Gd³ complexes with R=[Gd]/[3.AA]=1 for the right or left renal cortex (RC), the right or left pelvic cavity (PC) and the adrenal gland (AG).

FIG. 8 represents the measurement areas (renal cortex (RC), pelvic cavity (PC) and adrenal gland (AG)) used for FIG. 7 (R=right, L=left and BKG=background).

FIG. 9 represents the size distribution of complexes between the PEO₂ₖ-PVPA₁ₖ polymer and Gd³⁺ ions.

FIG. 10 shows the percentage of free Cu²⁺ ions (in gray) and free Gd³⁺ ions (in black) depending on pH from a solution of PEO_{6K}-b-PAA_{3K} complexes and Cu²⁺ and Gd³⁺ ions.

FIG. 11 shows the size distribution of complexes between the PEO₃ₖ-PVPA₆ₖ polymer and Gd³⁺/Eu³⁺/Cu²⁺ (⅓, ⅓, ⅓) ions at pH 7.34.

FIG. 12 shows the size distribution of complexes between Gd³⁺ ions, the PAA₃ₖ-PEO₆ₖ copolymer and the PAA₃ₖ-b-PDMA₇ₖ-coumarin fluorescent copolymer (10% by mass relative to the PAA₃ₖ-PEO₆ₖ copolymer).

FIG. 13 represents an emission spectrum of complexes between Gd³⁺ ions, the PAA₃ₖ-PEO₆ₖ copolymer and the PAA₃ₖ-b-PDMA₇ₖ-coumarin fluorescent copolymer at a percentage that can vary by mass.

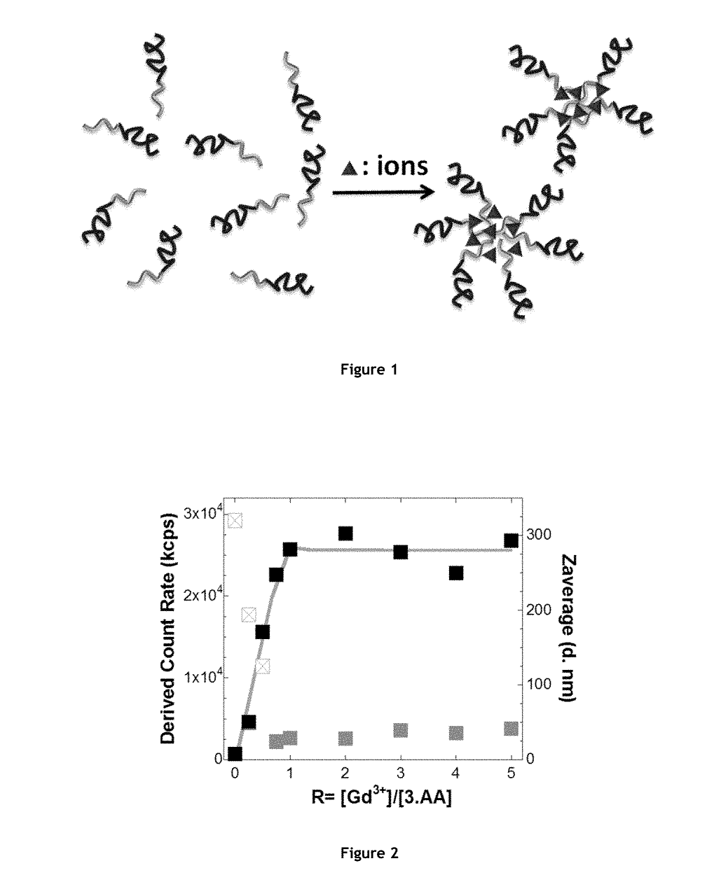

### EXAMPLES

For the preparation of complexes according to the invention, the recommended method is to work with a first series of experiments in which the chosen ions are mixed with the block polymers selected in the same aqueous solution and in variable concentrations. Analysis by an ad hoc technique (light scattering, fluorescence, UV-visible spectroscopy, etc.) of its solutions allows monitoring the formation of complexes depending on the ratio of the ion concentration to the block polymer concentration and thus selecting an optimal ratio for which all the ions are complexed by all the polymers. However, one can work with a higher concentration ratio (i.e., with an excess of ions) or lower concentration ratio (i.e., with an excess of block polymer):in these cases, the final solutions will be a mixture of complexes+ ions or free block polymers that will easily be eliminated by dialysis.

1. Complex Between the PEO_{6K}-b-PAA_{3K} Polymer and Gd³⁺ Ions

A 0.1% solution by mass of PEO_{6K}-b-PAA_{3K} polymer in water (corresponding to a concentration in acetic acid (AA) groups of [3.AA]=1·54·10⁻³ mol/L) is mixed with an aqueous solution of Gd(NO₃)₃ of concentration 1·54 mol/L.

The first complexation experiments (monitored, for example, by dynamic light scattering) show a break in slope (in the intensity of scattered light or the average size of objects observed) for a ratio of 1 between gadolinium and the concentration of 3 acetic groups (see FIG. 2).

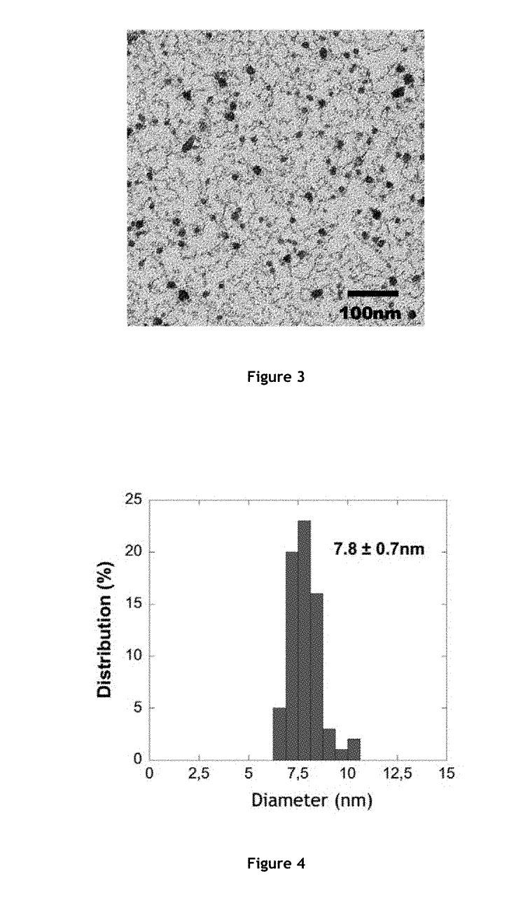

This ratio 1 is the preferential ratio used to form the complexes. Typically, 2 μL of gadolinium solution are added to 20 mL of the preceding polymer solution with stirring. The solution pH can then be adjusted if necessary by addition of an HCl or NaOH solution. Dynamic light scattering then shows the formation of complexes of about 10-15 nm in diameter. Electron microscopy confirms the size of these objects with a mean diameter of 8-10 nm for the charged-ion part (FIG. 3). The particle size distribution of the complexes is illustrated by FIG. 4.

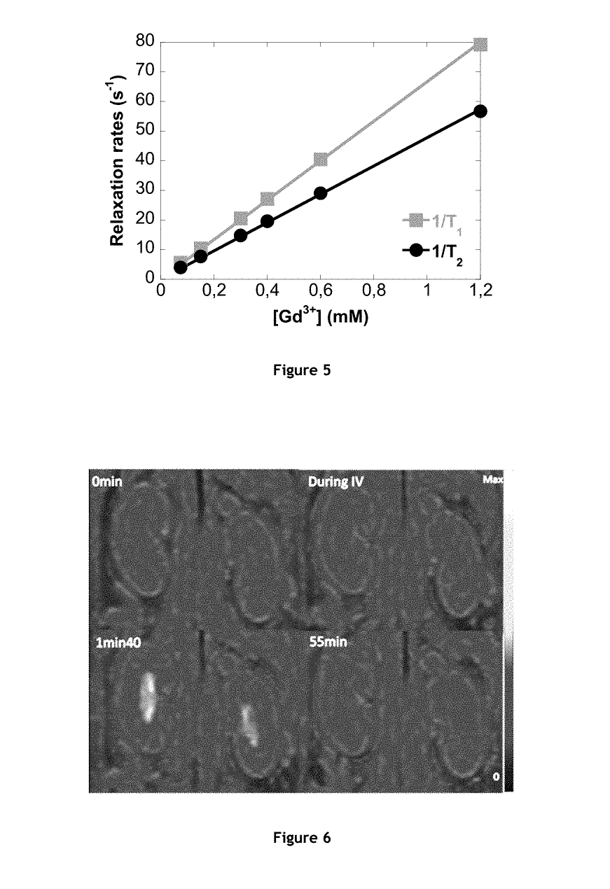

After dialysis (cut-off limit: 2,000 g·mol⁻¹) of this solution, the relaxivity measurements (at 25° C. and 1.4 T) give relaxivity values r₁=48 mM⁻¹·s⁻¹ and r₂=67 mM⁻¹·s⁻¹ (see FIG. 5).

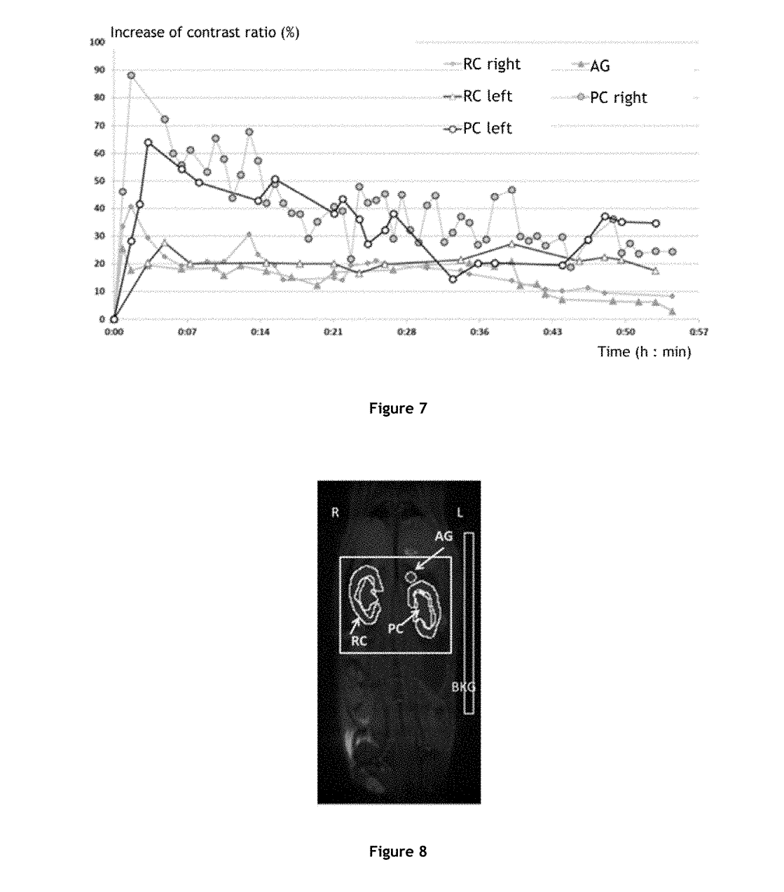

These complex solutions are used in MRI after adjustment of the ionic strength and the pH to render the solution compatible with physiological conditions. It is then injected intravenously. For example, measurements in rats (female Wistar rats of 190 to 240 g) are done by injection of 500 microliters of solution (concentration: 15 mmol/kg of Gd). The images and analysis of the contrast over time in the renal region of these rats are shown in FIGS. 6 and 7 (FIG. 8 represents the measurement areas in the rat).

2. Complex Between the PEO_{11K}-b-PAA_{3.7K} Polymer and Gd³⁺ Ions

2 μL of a 1·17 mol/L gadolinium solution are added to 20 mL of a PEO_{11K}-b-PAA_{3.7K} polymer solution at 0.1% by mass with stirring. Dynamic light scattering then shows the formation of complexes of about 10-15 nm in diameter.

3. Complex Between the PEO₂ₖ-PVPA₁ₖ Polymer and Gd³⁺ Ions

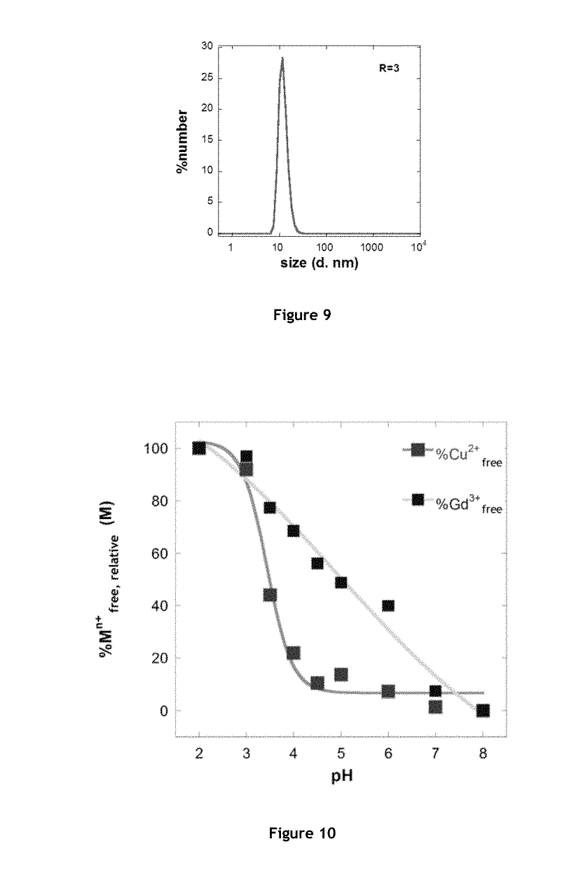

0.01 mol/L aqueous solutions of Gd(NO₃)₃.6H₂O and PEO₂ₖ-PVPA₁ₖ at 0.5 mass % are prepared beforehand and adjusted to pH=7. These solutions are then mixed and supplemented with deionized water to have a polymer concentration of 0.1 mass % and a Gd³⁺ concentration of about 0.15 mM. The [VPA unit]/[Gd³⁺] ratio is 1. The size (measured by dynamic light scattering) of the complexes formed is around 10 nm (see FIG. 9).

4. Synthesis of the Ternary System Containing Ln³⁺ Ions and (x)PAA-b-PEO/(1-x)PAA-b-PNIPAM (Ln=Eu³⁺ or Gd³⁺)

Aqueous solutions of Ln(NO₃)₃.xH₂O at 0.01 mol/L (Gd(NO₃)₃.6H₂O or Eu(NO₃)₃.5H₂O), PAA_{7.5k}-b-PEO_{22.5k} at 0.5 mass % and PAA₁₀ₖ-b-PNIPAM₂₄ₖ at 0.5 mass % are prepared beforehand and adjusted to pH=7. These solutions are then mixed in this order and supplemented with deionized water to have a polymer concentration of 0.1 mass % and a Ln³⁺ concentration of about 0.15 mM. The [acrylate unit]/[Ln³⁺] ratio is 3. For the ternary system containing Gd³⁺ ions and a mass fraction of PAA-b-PEO/PAA-b-PNIPAM=9, 306, 360 and 36 μL of the above-mentioned stock solutions of Gd(NO₃)₃, PAA_{7.5k}-b-PEO_{22.5k} and PAA₁₀ₖ-b-PNIPAM₂₄ₖ are respectively added and filled to 2 mL with 1300 μL of ionized water. The ionic strength of the solution can then be adjusted by addition of appropriate ions (NaCl for example) The system obtained has a hydrodynamic diameter of 90 nm at room temperature. In this system, the PNIPAM block confers a heat-sensitive nature to the assembly formed (in addition to the relaxivity properties related to the presence of Gd³⁺ ions). In fact, for temperatures greater than 32° C., the PNIPAM is dehydrated to become hydrophobic. Thus, the hydrodynamic diameter (evaluated from the distribution of the diameter by number) increases from 90 to 150 nm when the temperature goes from 25° C. to 45° C., to regain its initial value when the temperature returns to 25° C.

For the ternary system containing Ln³⁺ ions and a mass fraction of PAA-b-PEO/PAA-b-PNIPAM=9, 306, 360 and 36 μL of the above-mentioned stock solutions of Eu(NO₃)₃, PAA_{7.5k}-b-PEO_{22.5k} and PAA₁₀ₖ-b-PNIPAM₂₄ₖ are respectively added and filled to 2 mL with 1300 μL of ionized water. The system obtained is therefore both luminescent (presence of Eu³⁺) and heat sensitive (presence of PNIPAM).

### 5. Synthesis of Ternary System Containing Two Types of Ions and a PAA-b-PEO Block Polymer

Aqueous solutions of Gd(NO₃)₃.6H₂O and CuSO₄.6H₂O at 0.5 mol/L and PAA₃ₖ-b-PEO₆ₖ at 0.5 mass % are prepared beforehand and adjusted to pH=7.4 mL of PAA₃ₖ-b-PEO₆ₖ solution are added to a mixture of 46.2 μL of Gd(NO₃)₃.6H₂O solution and 46.2 μL of CuSO₄.6H₂O and 15.910 mL of deionized water. The pH of these solutions is adjusted between 2 and 8 by addition of concentrated 1 mol/L NaOH or HCl solutions. If the two ions are both completely complexed at pH 8, a reduction in pH leads to a differentiated release of the two ions (see FIG. 10). This release can be used to locally release ions of interest.

Other ternary systems based on zirconium and copper ions are obtained from similar proportions of aqueous solutions of Zr(NO₃)₄ and CuSO₄.6H₂O at 0.5 mol/L and PAA₃ₖ-b-PEO₆ₖ at 0.5 mass % prepared beforehand and adjusted to pH=7. For this system, a progressive release of Cu²⁺ ions when the pH is reduced is observed, while Zr⁴⁺ ions are held within the assembly regardless of the pH.

### 6. Synthesis of Quaternary System Containing Three Types of Ions and a PAA-b-PEO Block Polymer

Aqueous solutions of Gd(NO₃)₃.6H₂O, Eu(NO₃)₃.5H₂O and CuSO₄.6H₂O at, respectively, 5.14 mmol/L, 0.311 mol/L and 5.15 mmol/L and PAA₃ₖ-b-PEO₆ₖ at 0.1 mass % are prepared beforehand and adjusted to pH=7.261 μL of the PAA₃ₖ-b-PEO₆ₖ solution are added to a mixture of 700 μL of the Gd(NO₃)₃.6H₂O solution, 11.6 μL of Eu(NO₃)₃.5H₂O solution and 1049 μL of CuSO₄.6H₂O and 5 mL of deionized water. The pH of these solutions is adjusted between 1 and 8 by addition of concentrated 1 mol/L NaOH or HCl solutions.

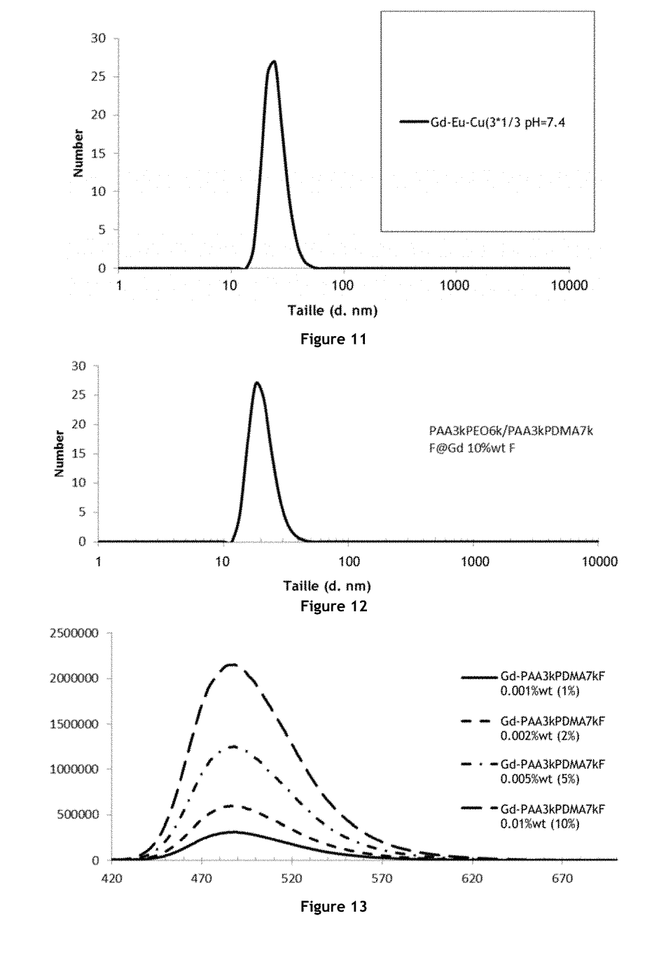

The complexes obtained are stable at pH>3.45 with a diameter of 23±3 nm (size measured by dynamic light scattering—see FIG. 11 for pH=7.34).

7. Synthesis of a Ternary System Containing Gd³⁺ Ions, a PAA₃ₖ-b-PEO₆ₖ Copolymer and a Fluorescent Copolymer (PAA₃ₖ-b-PDMA₇ₖ-Coumarin)

Complexes of Gd³⁺ ions, a PAA₃ₖ-b-PEO₆ₖ copolymer and a fluorescent copolymer (PAA₃ₖ-b-PDMA₇ₖ-coumarin), with different proportions of the two copolymers, were prepared from solutions of PAA₃ₖ-b-PEO₆ₖ and PAA₃ₖ-b-PDMA₇ₖ-coumarin at a concentration of 1%. For example, 10 μL of the PAA₃ₖ-b-PDMA₇ₖ-coumarin added to 990 μL of the PAA₃ₖ-b-PEO₆ₖ solution provide 1 mL of a mixture of the two polymers with a total concentration of 1% and a 99/1 concentration ratio between the two polymers.

The proportions by mass of the two copolymers used in this example are reported in the table below.

| Total polymer | | |
|---|---|---|
| concentration | % PAA₃ₖ-b-PEO₆ₖ | % PAA₃ₖ-b-PDMA₇ₖ-coumarin |
| 0.1% by weight | 99% | 1% |
| 0.1% by weight | 97.9% | 2.1% |
| 0.1% by weight | 95% | 5% |
| 0.1% by weight | 90% | 10% |

An aqueous solution of Gd(NO₃)₃.6H₂O at 0.1 mol/L and a mixture of polymers at 0.1 mass % are prepared beforehand and adjusted to pH=7.4 mL of the polymer solution are added to a mixture of 46.2 μL of the Gd(NO₃)₃.6H₂O solution and 15.95 mL of deionized water. Measurement by dynamic light scattering showed that regardless of the doping in fluorescent copolymer (from 1 to 10% of the total polymer composition), the size of the complexes formed remains constant with a diameter of about 20 nm (see FIG. 12 for the sizes of complexes at 10% fluorescent copolymer). The fluorescence spectra (in aqueous solution, 1 cm optical path curve, 414 nm excitation wavelength) were also recorded (see FIG. 13). As expected, the intensity of fluorescence increases with the percentage of fluorescent copolymer.

### 8. Formation of Complexes Between Gd³⁺ Ions and Different PAA-b-PEO Copolymers

Complexes between Gd³⁺ ions and different PAA-b-PEO copolymers were prepared. Aqueous solutions at 0.01 mol/L of Gd(NO₃)₃.6H₂O and PEOₓ-PAA_{y} at 0.5 mass % are prepared beforehand and adjusted to pH=7. These solutions are then mixed and supplemented with deionized water to have a polymer concentration of 0.1 mass % and a Gd³⁺ concentration such that the [PAA unit]/[Gd³⁺] ratio is 1. The sizes of the complexes obtained were measured by dynamic light scattering. The results obtained are reported in the table below.

| PAA-b-PEO | Particle diameters |
|---|---|
| PAA₃ₖ-b-PEO₆ₖ | 20 ± 2 nm |
| PAA₆ₖ₋b-PEO₁₁ₖ | 27 ± 4 nm |
| PAA_{6.5k}-b-PEO₆ₖ | 33 ± 2 nm |
| PAA_{7.5k}-b-PE0_{22.5k} | 12 ± 2 nm |

This experiment shows that it is possible to control the particle size by adjusting the length of the two blocks of the copolymer. At constant PEO molar mass, the size of the complexes increases when the molar mass of PAA increases. At constant PAA molar mass, the size of the complexes decreases when the molar mass of PEO increases.

### REFERENCES

Abraham et al. European Journal of Radiology 2008, 66, 200-207
Bouyer et al. Colloids and Surfaces A: Physiochem. Eng. Aspects 2003, 217, 179-184
Bottomley et al. Med. Phys. 1984, 11, 425-448
Oostendorp et al. Magn. Reson. Med. 2010, 64, 291-298
Sanson et al. Langmuir 2012, 28, 3773-3782
Tarasov et al. New J. Chem. 2013, 37, 508-514
Tseng et al. Biomaterial, 2010, 31, 5427-5435
US 2007/0154398

1.-16. (canceled)

17. A method for medical imaging comprising the administration to a subject in need of an effective dose of a complex between:
block polymers comprising a first hydrophilic block and a second block comprising at least one ionized function, and optionally carrying one or more additional chemical or biochemical groups or including a third block polymer, or mixtures of these polymers, and
ions comprising at least two positive charges chosen from among the elements Cu, Ga, Sr, Bi, Sc, Y, lanthanide, Pb, Tc, Zn, and Zr at different degrees of ionization and mixtures thereof,
said complex comprising the blocks comprising at least one ionized function in interaction with the ions at the center and the hydrophilic blocks at the periphery.

18. The method according to claim 17, wherein the hydrophilic block is a poly(ethylene oxide) (PEO) block, a poly(N-isopropylacrylamide) (PNIPAM) block, a polyvinylpyrrolidone (PVP) block, a polyvinylcaprolactam (PVCL) block, a polydimethylacrylamide (PDMA) block or a mixture thereof.

19. The method according to claim 17, wherein the block comprising at least one ionized function is a block comprising one or several —CO₂⁻, —O⁻, —PO₃²⁻, —OPO₃²⁻, —SO₃⁻, —OSO₃⁻ functions or a combination thereof.

20. The method according to claim 17, wherein the block comprising at least one ionized function is a poly(acrylic acid) (PAA) block, poly(vinylphosphonic acid) (PVPA) block, or a mixture thereof, in an ionized form.

21. The method according to claim 17, wherein the number average molar mass of the block polymer is comprised between 2,000 g·mol⁻¹ and 70,000 g·mol⁻¹.

22. The method according to claim 21, wherein the number average molar mass of the block polymer is comprised between 8,000 g·mol⁻¹ and 26,000 g·mol⁻¹.

23. The method according to claim 17, wherein the ions are chosen from among Cu²⁺, Eu³⁺, Gd³⁺, Tc⁵⁺, Zr⁴⁺ and mixtures thereof.

24. The method according to claim 23, wherein the ions are chosen from among Cu²⁺, Gd³⁺ and mixtures thereof.

25. The method according to claim 17, wherein the medical imaging is magnetic resonance imaging (MRI), fluorescence imaging or scintigraphic imaging, including positron emission tomography (PET) or alpha emission imaging.

26. The method according to claim 17, for visualizing a pathological structure.

27. The method according to claim 26, wherein the pathological structure is a tumor.

28. A physiologically-acceptable composition comprising, in a physiologically-acceptable medium, a complex as defined in claim 17,
with the exception of:
a complex between Zn²⁺ ions and poly(acrylic acid)-polyacrylamide (PAA-PAM) diblock polymers in the ionized form for which the poly(acrylic acid) block has a number average molecular weight of 1,000, 3,000, or 5,000 g·mol⁻¹ and the polyacrylamide block has a number average molecular weight of 10,000, 15,000, or 30,000 g·mol⁻¹ respectively,
a complex between La³⁺ ions and poly(acrylic acid)-polyacrylamide (PAA-PAM) diblock polymers in an ionized form for which the polyacrylamide block has a number average molecular weight of 10,000 g·mol⁻¹ and the poly(acrylic acid) block has a number average molecular weight of 1,000, 3,000 or 6,000 g·mol⁻¹; or the polyacrylamide block has a number average molecular weight of 11,100 g·mol⁻¹ and the poly(acrylic acid) block has a number average molecular weight of 2,800 g·mol⁻¹; or the polyacrylamide block has a number average molecular weight of 30,000 g·mol⁻¹ and the poly(acrylic acid) block has a number average molecular weight of 3,000 or 5,000 g·mol⁻¹; or the polyacrylamide block has a number average molecular weight of 60,000 g·mol⁻¹ and the poly(acrylic acid) block has a number average molecular weight of 5,000 g·mol⁻¹, and
a complex between La³⁺ ions and poly(acrylic acid)-poly(2-hydroxyethyl acrylate) (PAA-PHEA) diblock polymers in the ionized form for which the poly(acrylic acid) block has a number average molecular weight of 1,900, or 2,800 g·mol⁻¹ and the poly(2-hydroxyethyl acrylate) block has a number average molecular weight of 8,200, or 11,100 g·mol⁻¹, respectively.

29. The composition according to claim 28, adapted to parenteral administration or to oral administration.

30. A complex between:
block polymers comprising a first hydrophilic block and a second block comprising at least one ionized function, and optionally carrying one or more additional chemical or biochemical groups or including a third block polymer, or mixtures of these polymers, and
ions comprising at least two positive charges chosen from among the elements Cu, Ga, Sr, Bi, Sc, Y, lanthanide such as Eu or Gd, Pb, Tc, Zn, and Zr at different degrees of ionization and mixtures thereof,
said complex comprising the blocks comprising at least one ionized function in interaction with the ions at the center and the hydrophilic blocks at the periphery,
with the exception of:
complexes between Zn²⁺ ions and poly(acrylic acid)-polyacrylamide (PAA-PAM) diblock polymers in the ionized form for which the poly(acrylic acid) block has a number average molecular weight of 1,000, 3,000, or 5,000 g·mol⁻¹ and the polyacrylamide block has a number average molecular weight of 10,000, 15,000, or 30,000 g·mol⁻¹ respectively,
complexes between La³⁺ ions and poly(acrylic acid)-polyacrylamide (PAA-PAM) diblock polymers in an ionized form for which the polyacrylamide block has a number average molecular weight of 10,000 g·mol⁻¹ and the poly(acrylic acid) block has a number average molecular weight of 1,000, 3,000 or 6,000 g·mol⁻¹; or the polyacrylamide block has a number average molecular weight of 11,100 g·mol⁻¹ and the poly(acrylic acid) block has a number average molecular weight of 2,800 g·mol⁻¹; or the polyacrylamide block has a number average molecular weight of 30,000 g·mol⁻¹ and the poly(acrylic acid) block has a number average molecular weight of 3,000 or 5,000 g·mol⁻¹; or the polyacrylamide block has a number average molecular weight of 60,000 g·mol⁻¹ and the poly(acrylic acid) block has a number average molecular weight of 5,000 g·mol⁻¹, and
complexes between La³⁺ ions and poly(acrylic acid)-poly(2-hydroxyethyl acrylate) (PAA-PHEA) diblock polymers in the ionized form for which the poly(acrylic acid) block has a number average molecular weight of 1,900, or 2,800 g·mol⁻¹ and the poly(2-hydroxyethyl acrylate) block has a number average molecular weight of 8,200, or 11,100 g·mol⁻¹, respectively.

31. The complex according to claim 30, wherein the hydrophilic block is a poly(ethylene oxide) (PEO) block, a poly(N-isopropylacrylamide) (PNIPAM) block, a polyvinylpyrrolidone (PVP) block, a polyvinylcaprolactam (PVCL) block, a polydimethylacrylamide (PDMA) block or a mixture thereof.

32. The complex according to claim 30, wherein the block comprising at least one ionized function is a poly(acrylic acid) (PAA) block, poly(vinylphosphonic acid) (PVPA) block, or a mixture thereof, in an ionized form.

33. The complex according to claim 30, wherein the ions are chosen from among Cu²⁺, Eu³⁺, Gd³⁺, Tc⁵⁺, Zr⁴⁺ and mixtures thereof.

34. The complex according to claim 33, wherein the ions are chosen from among Cu²⁺, Gd³⁺ and mixtures thereof.

35. A process for the preparation of a complex according to claim 30 comprising the complexation reaction between:
at least one block polymer comprising a first hydrophilic block and a second block comprising at least one ionized function or ionizable function, and optionally carrying one or more additional chemical or biochemical groups or including a third block polymer, and
at least one salt of an ion comprising at least two positive charges chosen from among the elements Cu, Ga, Sr, Bi, Sc, Y, lanthanide, Pb, Tc, Zn, and Zr at different degrees of ionization.

36. The process according to claim 35, wherein the complexation reaction is performed in water as a solvent.
